(19) United States
(12) Patent Application Publication
(10) Pub. No.: US 2008/0228200 A1
(43) Pub. Date: Sep. 18, 2008

(54) CLOSURE AND RECONSTRUCTION IMPLANTS AND THE APPARATUS FOR DELIVERY THEREOF

(76) Inventor: Clinton Baird, Abingdon, MD (US)

Correspondence Address: CERMAK KENEALY & VAIDYA LLP, 515 E. BRADDOCK RD, SUITE B, ALEXANDRIA, VA, 22314, US

(21) Appl. No.: 12/049,359

(22) Filed: Mar. 16, 2008

Related U.S. Application Data
(60) Provisional application No. 60/895,287, filed on Mar. 16, 2007.

(51) Int. Cl. A61B 17/10; A61B 17/04

(52) U.S. Cl. 606/139; 606/144

(57) ABSTRACT

A system and implant is useful for the minimally invasive closure of dura, bone, and/or ligementous defects. The delivery mechanism allows for the novel deployment of a novel closure device. The implant device with three components, an internal anchor, disc, and retaining ring, is loaded into a delivery system that allows minimally invasive closure and/or reconstruction of anatomical defects. Specifically, the device is designed to be released from the delivery system into narrow spaces of anatomical structures created during surgery. Inner and outer members are connected and locked together in situ which allows for additional anatomical manipulations to take place as required for reconstruction and closure. This allows each component, particularly the inner member, to be tailored to the anatomy.

This application claims priority under 35 U.S.C. § 119 to U.S. Provisional Application No. 60/895,287, filed 16 Mar. 2007, the entirety of which is incorporated by reference herein.

### BACKGROUND

1. Field of Endeavor

The invention relates to minimally invasive closure implants and the apparatus and method for delivery of the implant devices. The devices are intended for closure or reconstruction of connective or supporting structures (e.g. dura, bone, annulus, ligaments) surrounding or supporting neural or neurovascular anatomy.

2. Brief Description of the Related Art

Neurosurgical procedures, both cranial and spinal, require repair of bone and soft tissue defects (bone, dura, annulus, ligaments etc) created during a surgical intervention, trauma, or other pathological processes. The proper repair of such defects is crucial to the successful outcome of the operation. Current methods for closing soft tissue defects include direct sutured closure, graft patched sutured closure with use of autologous, allogeneic, xenograft and/or synthetic grafting materials, tissue sealants, and occlusive packing with fat or other materials. U.S. Pat. No. 5,997,895 to Narotam, et al. describes traditional onlay and suturable dural grafts.

Delivery of and securing dural grafts, annular closure devices, bone closure devices or other soft tissue closure or reconstruction around neural or neurovascular elements through minimally invasive techniques currently does not result in satisfactory outcomes. These techniques continue to have a significant amount of associated cerebrospinal fluid leakage, soft tissue herniation, or recurrent disc fragment extrusion. Cerebrospinal fluid leakage outside of the cranial or spinal cavity significantly increases the risk for complications such as meningitis, wound infection, poor wound healing, neurological injury, pseudomeningocele, pneumocephalus, rhinorrhea, and/or death.

The use of minimally invasive surgical techniques in neurosurgery further limits the ability to directly repair dural openings/defects at the time of closure, bone defects (burr holes, craniotomies, craniectomies) or annular defects. Furthermore, the time required for traditional closure increases the risk associated with longer operations and associated iatrogenic injury. This limited ability to repair surgically created dura, bone, or ligamentous defects is a barrier to the progress of minimally invasive neurosurgery. As a result, many surgeons continue to use more invasive traditional approaches in which closure can be performed more directly. Those who offer minimally invasive approaches are forced to use less effective packing techniques and occasionally difficult to employ suturing techniques to close the dural, bone, or ligament defect. The development of a device that can be delivered through minimally invasive techniques could allow for a more effective method of reconstruction and closure. This is likely to remove one of the major barriers to minimally invasive neurosurgery, namely cerebrospinal fluid leakage and inadequate bone and ligament repair.

In minimally invasive spinal procedures, tubular instruments allow operations through minimal access openings and allow surgical decompression and placement of spinal hardware. However, there are currently no devices that enable the controlled delivery of small dural closure devices through minimal access surgery. Further, available ligament closure devices (annulus closure) do not allow adequate control of the implanted device during implantation. The method of closure implant delivery described herein may allow for safe and effective closure or reconstruction in both spinal and cranial interventions.

### SUMMARY

According to a first aspect of the invention, a system for closure of anatomical openings in a patient comprises an anchor including a first locking element extending along a longitudinal direction and at least two lateral elements extending at least partially laterally from the longitudinal direction, a flexible sealing disc including a sealing membrane and a hole through said disc sized to permit passage of the first locking element therethrough, and a retaining ring including a second locking element configured and arranged to receive the first locking element and lockingly retain the ring to the anchor.

According to another aspect of the present invention, a method for closing an anatomical defect in a patient, the defect including a hole with a lateral dimension, the method comprises inserting an anchor through said hole, a portion of the anchor extending back through the hole, and attaching a seal to said anchor portion, said seal extending laterally farther than said defect hole lateral dimension.

Still other aspects, features, and attendant advantages of the present invention will become apparent to those skilled in the art from a reading of the following detailed description of embodiments constructed in accordance therewith, taken in conjunction with the accompanying drawings.

### BRIEF DESCRIPTION OF THE DRAWINGS

The invention of the present application will now be described in more detail with reference to exemplary embodiments of the apparatus and method, given only by way of example, and with reference to the accompanying drawings, in which:

FIG. 1 illustrates an exemplary embodiment of an internal anchor of the implant device.

FIG. 2 illustrates a perpendicular cross section of the tubular component of the internal anchor illustrated in FIG. 1.

FIG. 3 illustrates an oblique cross section of the internal anchor of FIG. 1.

FIG. 4 illustrates an internal view of an exemplary embodiment of a disc according to the invention.

FIG. 5 illustrates an external view of the disc of FIG. 4.

FIG. 6 illustrates an internal view of the disc of FIG. 4, in cross section.

FIG. 7 illustrates an exemplary embodiment of a retaining ring according to the invention.

FIG. 8 illustrates an exemplary embodiment of an assembled implant, in cross section.

FIG. 9 illustrates an exemplary embodiment of a delivery system.

FIG. 10 illustrates an exemplary mechanism for controlled advancement of the inner and outer pushers.

FIG. 11 illustrates an exemplary embodiment of a mechanism for suture tensioning to engage the internal anchor with the delivery system.

FIG. 12 illustrates a cross sectional view of the distal end of the delivery device internal components.

FIG. 13 illustrates the dynamic component of the manipulating pusher.

FIG. 14 (a) shows the devices in an exemplary anatomical setting, while FIG. 14(b) shows an enlarged view of the distal end of an exemplary system and anchor.

FIG. 15 illustrates distal end portions of an exemplary system, with portions broken away.

### DETAILED DESCRIPTION OF EXEMPLARY EMBODIMENTS

Exemplary devices embodying principles of the present invention, intended for reconstruction, augmentation or other support of neural structures and the surrounding elements (e.g. dura, bone, ligaments etc), are designed to allow safe and controlled deployment in the narrow anatomical spaces around the critical neuroanatomical structures enclosed by dura, bone, and ligaments.

One exemplary embodiment of a device includes a plurality of elongated tubes (cannula) having a plurality of dimensions, diameters, materials, and deformability. The tubes are arranged as to allow for the controlled pushing, pulling, and/or other movement of implant devices aimed at bone, dural, or ligamentous closure adjacent to the nervous system. The plurality of tubes, pushers, stoppers, and guide wires allow for the independent control of inner and outer components of various unrelated closure devices. The device allows the minimally invasive, cannulae-based delivery of implants required to close dural, bone or ligamentous defects.

The invention also relates to the minimally invasive closure of dural, bone, or ligamentous defects adjacent to the nervous system. The delivery mechanism allows for the novel deployment of a novel dural closure device, bone reconstruction/closure device, and ligementous closure device. The device is designed to release one or more implants from the delivery cannula into narrow spaces around the nervous system in a controlled, minimally invasive fashion, thus allowing deployment of a closure or reconstruction implant and, thus, closure of dural, bone, or ligamentous defects. This can be critical for deployment of implants designed for closure of bone, dura, or ligaments within or adjacent to neurological and neurovascular anatomic structures.

In general terms, an inner framework may include a simple bioresorbable plate, strut or plurality of struts with a central hub secured to the plate or strut member. The plate or strut can be deployed outside of the defect, and then be positioned within the defect. The method of deployment allows for minimally invasive deployment and low profile precisely controlled final positioning so as to encourage safe and effective closure or reconstruction adjacent to neural or neurovascular anatomy. After positioning of this inner component, the outer disc framework can be deployed the same as for any other outer framework described herein.

Delivery systems embodying principles of the present invention advantageously include a series of cannula, pushers, guiding tubes, and tension modulating sutures and/or wires to deploy the device or anchor in place. An outer guide delivery cannula that is relatively rigid and allows positioning of the device in proximity to dural, bone, or ligamentous structures. Within the outer delivery cannula or guide t are a plurality of pushers, sliders, delivery tubes, guide wires, and or tension modulating sutures/wires, which allow for the independent and controlled delivery of various implants. Specifically, it includes an outer implant pusher and an outer implant slider. This allows for pushing and sliding of the outer closure component to be advanced into position. The outer pusher has a recessed end to allow for the holding and pushing of implant devices onto an inner member. The inner pusher advances the inner device out of the outer delivery cannula. The inner pusher can be a tube, wire, rod, square, triangular pushing component within or outside of the shaft of the outer pusher and outer slider. Within the inner pusher, the inner guide cannula or wire is positioned. This allows for precise manipulation of the inner member of an implant required for safe and controlled positioning adjacent to the nervous system. The outer cannula, outer pusher, inner pusher, sliders, guide collars, guide cannula, stop collars, or guide rods may be arranged in a plurality of positions relative to one and other. There can be slots fashioned in the tubes in a plurality of dimensions and orientations to allow for the independent or dependent movement of one member with respect to another. Tension modulating sutures control the tension applied to the anatomic structures during device deployment. Positioning of the outer guide cannula, advancement of the semi-rigid inner deployment guide wire, expansion of the device, and manipulation of the tension modulating sutures is advantageously performed under direct endoscopic or microscopic visualization.

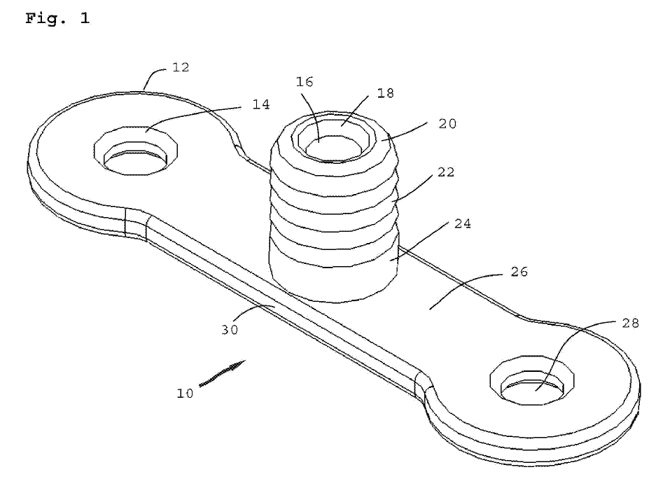

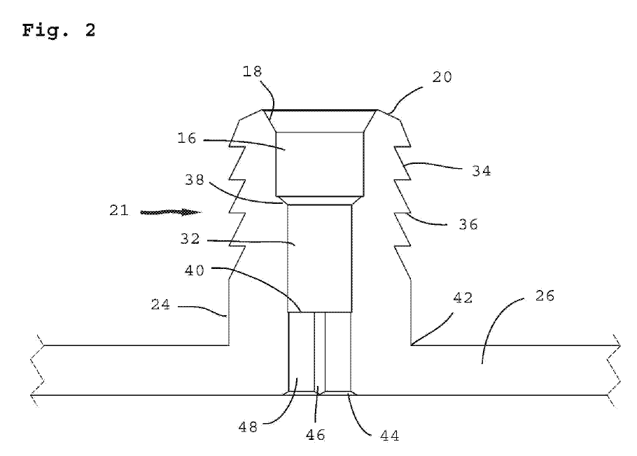

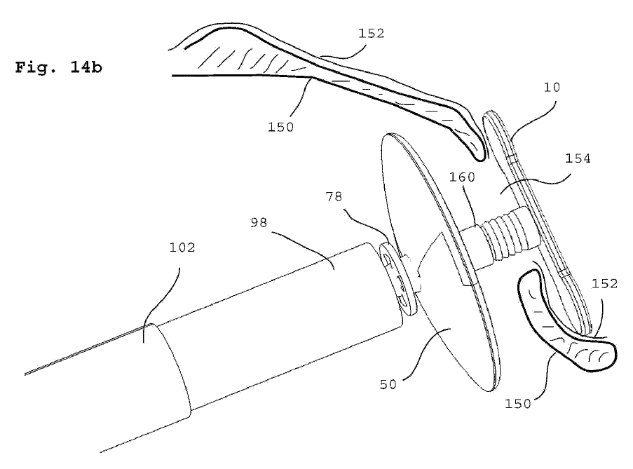

FIG. 1 illustrates an exemplary embodiment of an internal anchor 10 of a closure device embodying principles of the invention. The anchor 10 includes a planar component (plate) 26 which is perpendicular to a tubular docking hub 21. The planar component 26 is shown here as generally rectangular in shape, although other shapes, such as square, round, trapezoidal, or other irregular shapes, could be employed. The docking hub 21 as shown here is cylindrical in shape; alternatively, other shapes, such as square, round, trapezoidal, or other irregular shapes, could be employed. The internal anchor 10 has straight edges 30 that terminate in smooth, curved ends 12. Alternatively, the internal anchor edge 30 could be of variable shape, including an edge that is corrugated, sinusoidal, round, or of irregular angles. The planar component 26 of the internal anchor 10 is embodied as a linear dimension perpendicular to the docking hub base 24. Potential favorable alternatives include tangential, curved, or irregular dimensions relative to the central axis of the docking hub 21. Such alternatives are favorable to fit to various anatomy as shown in FIG. 14(b). By way of a non-limiting example, the thickness of the internal anchor in this embodiment is 0.75 mm, though it could be of any dimension that is compatible with the human anatomy. The intent of the anchor 10 is to create a structure which will capture the disc 50 against the desired anatomy. As such the anchor could be fashioned after a variety of additional structures including at least a balloon, an expandable sponge-like material, or retaining button or plug.

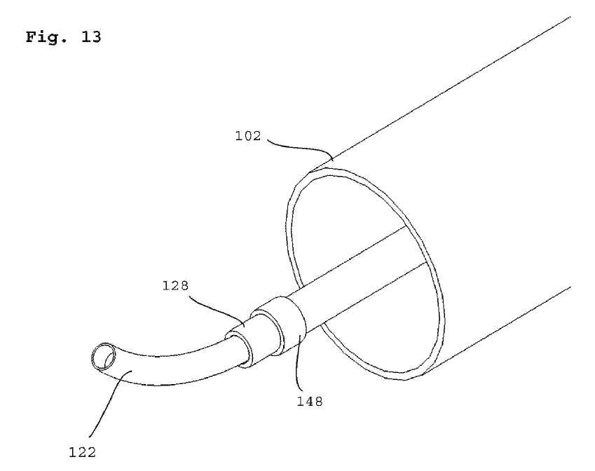

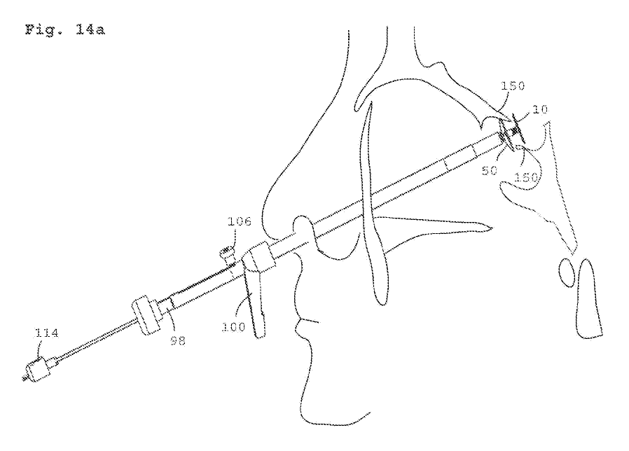

The docking hub base 24 is contiguous with a locking portion of the docking hub 21, shown here as a ratchet teeth 22. As detailed further below, the docking hub 21 engages with the internal manipulator 122 in the internal manipulator docking sleeve surface 32 and the internal pusher 128 in the inner pusher docking sleeve surface 16 (shown in FIGS. 12, 13). To favor guiding of the internal manipulator and internal pusher into the docking sleeves of the docking hub 21, the docking hub is chamfered on its internal 18 and external 20 entrance surfaces. The curved ends 12 of the planar portion 26 contain center through holes 28 with chamfered edges 14. The through holes 28 shown here are circular in shape, but could be of any shape. The inclusion of holes 28 in plate or planar portion 26 creates a favorable environment for tissue in growth, when the device is implanted in vivo. Further, other alternatives to the holes 28 include divits, pitting, or altering the courseness of the implant surface.

In some exemplary embodiments of systems of the present invention, the inner component of the device has a single internal anchor and a single docking hub and locking mechanism. Alternatively, the inner anchor 10 could have a plurality of planar components 26 projecting from the center axis of a docking hub, either in the same geometric plane or stacked upon one and other in geometric separate planes, can have a plurality of docking hubs, and can be of variable dimensions. Furthermore, the embodiment illustrated herein shows the planar potion of the internal anchor fixed relative to the docking hub. It may create favorable implantation performance to have one or a plurality of internal anchor plates (planar components) freely movable about a single or a plurality of docking hubs, such as by a snap-fit configuration or the like.

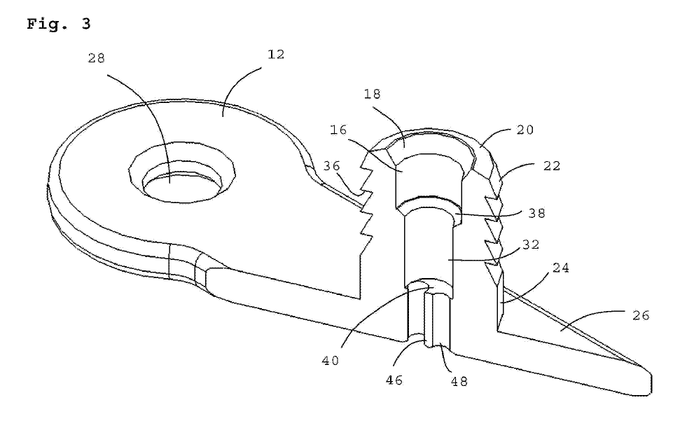

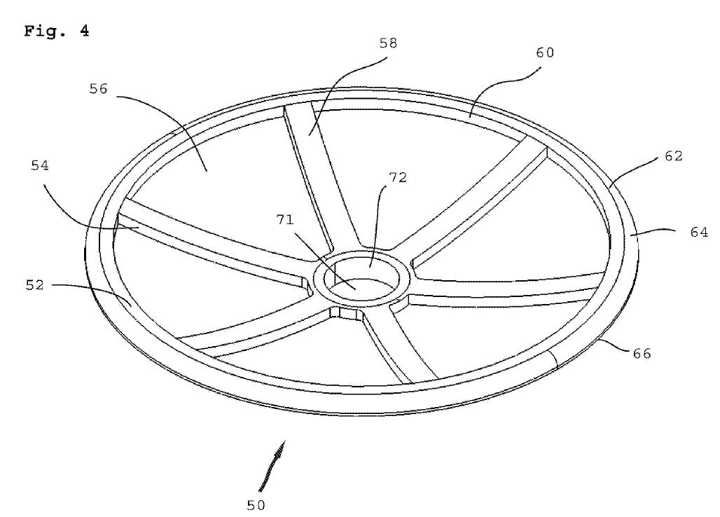

FIGS. 2 and 3 shows a cross section of an example of the docking hub 24 and the internal anchor plate 26 with an intersecting angle 42 of 90 degrees. This angle could be varied to create a favorable advantage of the seal between the outer disc through hole 71 (see FIG. 4) and the internal anchor docking hub 24. The internal manipulator docking sleeve 32 and the internal pusher docking sleeve 16 allow the device to interact and dock with a delivery system, as described in greater detail below. The internal manipulator (122) stop 40 prevents further advancement of the internal manipulator at a defined depth while the inner pusher (128) stop 38, here embodied as a frustoconical shoulder, prevents further advancement of the internal pusher. The docking sleeves are shown as cylindrical in shape, though other alternative shapes, including at least square, rectangular, star, trapezoidal, hexagonal, or irregular, can be used. Alternatively, the docking site could be modified to various shapes, depths, and angles. The size and shape of the docking sleeves is such as to allow engagement with the delivery system. Contiguous with the manipulator docking sleeve are the suture retention through holes 48, shown here perpendicular to the internal anchor plate and parallel to the docking hub 24. The suture retention through holes 48 preferably have chamfered edges 44 on the entry side 46 to allow for ease of suture insertion. The suture retention through holes 48 allow a retention suture to be passed through the internal anchor 10, which allows the retention suture to be used to create force to seat the docking sleeves onto the delivery system upon deployment of the internal anchor 10 and to create force against the desired anatomy. The retention suture can then be removed or left in place. Alternatively, the retention suture could be embedded into the internal anchor 10 at the time of manufacturing. Furthermore, the docking mechanism could be modified to include different mechanisms, including at least a docking pin and ball-and-socket docking mechanisms, as described in the aforementioned U.S. provisional patent application. Alternatively, the suture retention through holes 48 could be of other angles relative to the internal anchor and docking hub.

An exemplary locking mechanism includes a series of ratchet teeth 22 on the exterior of the docking hub 21, with an inclined sliding surface 34 and a locking surface 36 for locking of retaining ring pawls 82 (see FIG. 7). The locking mechanism demonstrated here embodies a ratchet mechanism that allows variable compression of the outer disc 50 (FIG. 4) onto the internal anchor 10. Advancement of the outer pusher 98 allows for ratcheting of the retaining ring onto the ratchet teeth, for locking of the device together, as detailed below. The chamfered edge 20 on the docking hub 24 creates a favorable geometry for the advancement of the retaining ring 78 onto the locking mechanism. Alternative locking mechanisms include, but are not limited to, rivets of various configurations, latch locking, suture retention, and/or incorporation of the pawls in to the central hub 72 of the disc 50.

FIG. 4 illustrates an exemplary embodiment of an outer closure disc, 50. Disc 50 contains the center hub 72 with a center through hole 71. The disc 50 is designed to create a closing seal of various anatomical defects. The inner 52 and outer 64 rims are rounded to allow for creation of a seal against the anatomy in which the disc 50 in implanted. The disc 50 includes spokes or struts 58 which have side walls 54 contiguous with a disc membrane 56 which extends between each of the struts and between the rim 52/64 and the hub 72, forming a sealed structure. The struts 58 are also contiguous with the disc rim 60 and the center hub 72. The inner aspect of the disc rim 60 and the outer aspect of the central hub 72 are preferably orthogonal to the disc struts, but alternatively can be formed at alternative angles. Alternative angles may favorably affect the dynamic characteristics of the disc 50. The plurality of struts 58 radiate from the central hub 72 mutually spaced at an angle of 60 degrees between them, for the exemplary embodiment in which there are six struts. Alternatively, fewer or more struts may be employed; while the struts are advantageously uniformly spaced around the disc 50, they may be non-uniformly spaced. The disc can be of other shapes including but not limited to oval, square, rectangular, trapezoidal, or other geometric shapes designed to close or reconstruct defects in the anatomy. Alternative shapes may also create a favorable advantage for the deployment and forces applied by the disc.

The disc struts 58 follow the same radius of the arc as the disc membrane outer surface 68 and are raised above the inner membrane surface 56 by a variable radius of the arc. Alternatively, the disc struts 58 could protrude from the outer or inner surfaces of the disc membrane 56 by a variable distance along the radius of the arc or independent of the radius of the arc. The disc struts 58 are arranged as a plurality of struts around the central hub separated by 60 degrees, linear in direction, and orthogonal to the central hub and disc rim. Alternatively the plurality of struts could vary in number, the shape can vary from rectangular, square, circular, oval, or an irregular shape, the orientation of the struts in the plane perpendicular to the central hub could vary from linear, curvilinear, sigmoid, or irregular, and the relationship of the struts to the central hub and disc rim could alternatively be non-orthogonal or irregular. Further, as shown here the struts 58 are formed from the same mold as the entire disc 50 as a monolithic structure. Alternatively, however, the struts could be manufactured separately and joined to the hub 72, rims, and/or disc membrane 56 in separate steps. Further the disc can function without struts by varying the thickness and/or materials of the disc membrane 56. All of these alternatives could create a favorable variation in the modulus of elasticity of the struts and/or disc membrane.

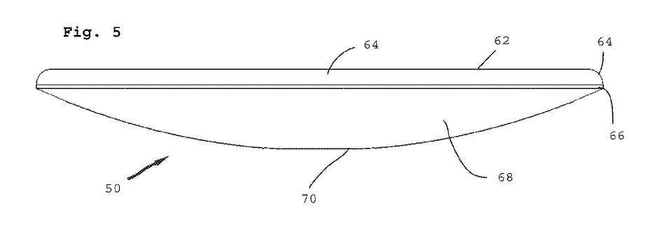

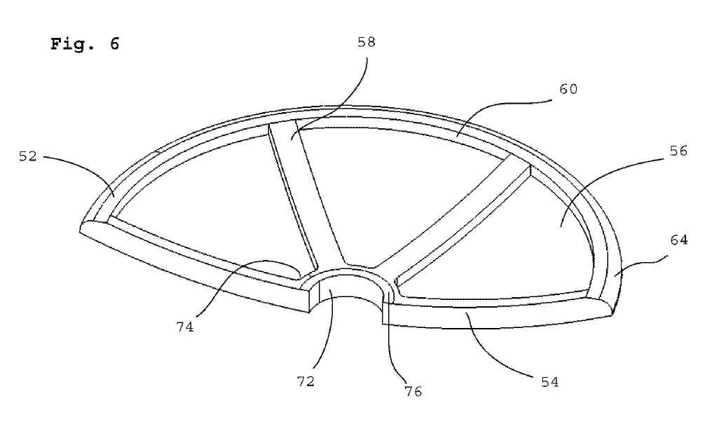

FIG. 5 illustrates the disc 50 from an outer view, perpendicular to the long axis of the central hub 72. The outer disc membrane 68 (and struts 58, not illustrated in FIG. 5) diverges from the outer docking surface 70 of the central hub 72 by a radius of the arc that can be varied. The membrane 56 terminates at the outer membrane rim 66 which is contiguous with the disc rim 64. This view illustrates the overall arc of the disc and the disc rim 62 perpendicular to the long axis of the central hub 72. Alternatively, the disc rim 62 and the outer docking surface of the central hub could be at variable orientation relative to the long axis of the central hub 72. This could create a disc rim 62 that is in the same geometric plane as the central hub 72 making the device flat or variable radii of the arc could be fashioned such that the disc rim 62 and central hub 72 are in different geometric planes.

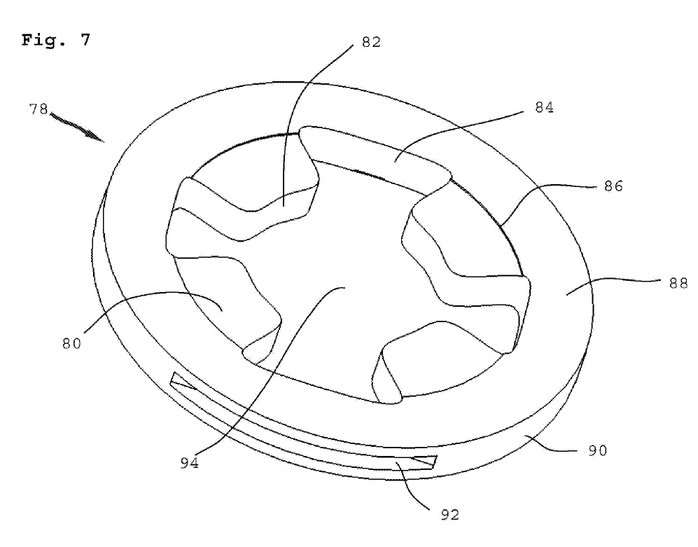

FIG. 6 illustrates the inner surface of the disc 50 in cross section through the struts 54 showing the relationship of the struts to the central hub 72, disc membrane 56, and disc rim 60. The inner surface 76 of the central hub 72 shown here is perpendicular to the long axis of the central hub 72. The strut diverges from this inner surface 76 of the central hub to follow a defined radius of the arc which converges on the disc rim 60. The intersection of the struts 58 with the outer rim of the central hub 74 is shown as rounded. Alternatively, the central hub could lack a perpendicular inner surface 76 with the struts radiating from the long axis of the central hub at a defined radius of the arc relative to the central axis of the central hub. Further the diameter of the inner surface 76 of the central hub could be varied. Additionally, the intersection of the struts 58 with the outer rim of the central hub 74 could be of variable angles. These alternatives modify the forces of the struts on the central hub. Such modification could be favorable for the deployment of the disc and closure of the anatomical defect. According to yet another exemplary embodiment, the disc 50 can be formed without struts, and the membrane 56 is constructed with sufficient strength to provide the needed rigidity to the disc. According to yet another embodiment, the disc 50 and the anchor 10 are constructed such that the anchor is stiffer than the disc, so that when the anchor and disc are implanted in a patient, the disc deforms more than the anchor when the two elements are compressed together with the retaining ring 78, thus causing the disc to better seal against the anatomical structures around the defect that is to be sealed.

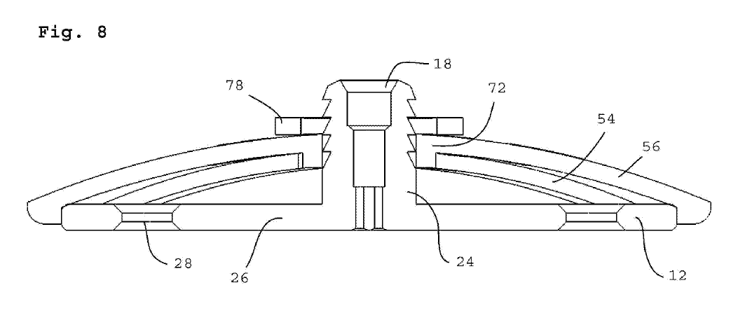

FIG. 7 illustrates an exemplary embodiment of a retaining ring, 78, with a center through hole 94 that is sized and configured to engage around the docking hub 24 of the internal anchor 10. The ratchet teeth 22 engage on the pawls 82 of the retaining ring 78, when the ring is pushed over the docking hub. Retaining ring pawls 80 are optionally grooved 86 or otherwise flexible to allow for movement of the pawls. The flat surface 88 of the retaining ring docks on the outer docking surface 70 of the central hub 72, while the opposite surface of the retaining ring and the ring surface 90 engage with the outer pusher 98 to advance the retaining ring 78, compressing the outer disc 50 onto the internal anchor 10, as illustrated in FIG. 8. When thus assembled together, a closing seal is created at the anatomical defect at which the device is implanted. The retaining ring 78 has two removal grooves 92 opposite to each other in the outer surface 90 which allow the engagement of a removal instrument (not illustrated) for forced removal of the engaged pawls with the ratchet teeth 36, by compression and distortion of the ring. Alternatively, the disc 50 could lock onto the internal anchor 10 by alternative mechanisms including, but not limited to, at least a rivet mechanism with direct locking of the internal anchor 10 to the disc 50, a suture tied mechanism, or a rotatory tongue-and-groove mechanism.

FIG. 8 illustrates a cross-sectional view of the exemplary anchor 10, outer closure disc 50, and retaining ring 78 assembled together. The anchor 10 is positioned with the docking hub 24 extending into the through hole 71, with the ratchet teeth 22 optionally bearing against the inner surface of hole to assist in holding the two structures together. As illustrated in FIG. 8, the anchor 10 is positioned on the ‘inner’ side of the closure disc 50, that is, on the concave side of the dome-shaped disc. The ring 78 is positioned on the opposite side of the closure disc 50, that is, on the convex side, with the ratchet teeth 22 extending through the hole 94 and engaging with the pawls 80, 82. Thus assembled, the retaining ring 78 holds the anchor 10 to the outer closure disc 50, with the outermost portions of the curved ends 12 and a portion of the planar surface 26 ‘inside’ an anatomical defect, and the inner surface of the disc 50 ‘outside’ an anatomical defect such that ‘closure’ is achieved. While FIG. 8 illustrates the length between the two ends 12 of the anchor 10 being slightly smaller than the diameter of the disc 50, the anchor can alternatively be the same size or larger than the disc, depending on, e.g., the size of the anatomical defect or hole that is to be sealed.

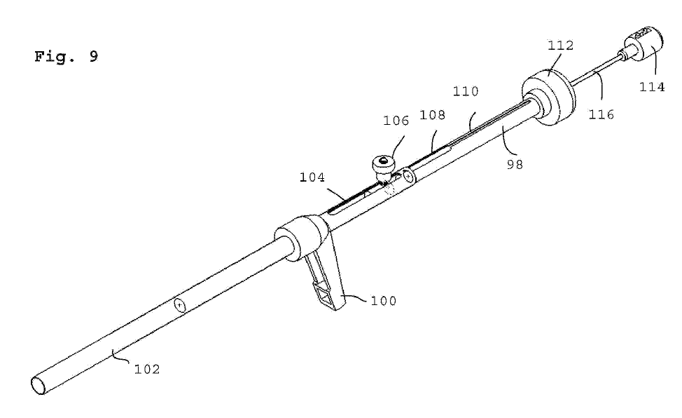

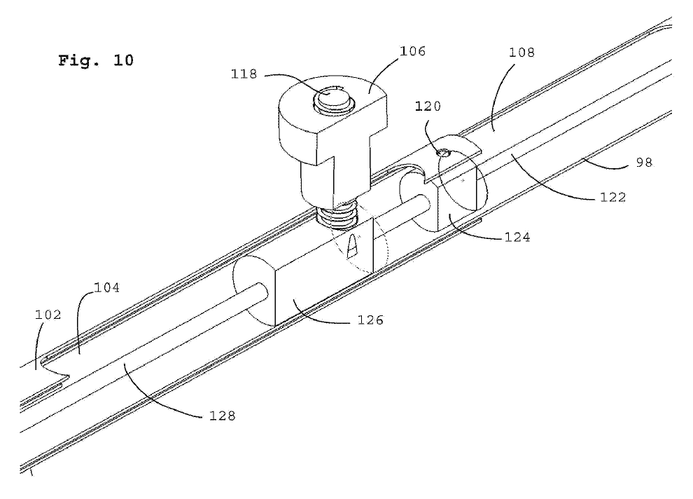

Now referring to FIG. 9, an assembled delivery system is illustrated in an ‘implant loaded’ configuration. Shown here, the system includes an outer delivery cannula 102 secured to the delivery system handle 100, an outer pusher 98 positioned in part within the outer delivery cannula 102, with an outer pusher knob 112 at its proximal end to allow advancement of the outer pusher 98 within the delivery cannula 102. Slots 104 cut in a portion of the delivery cannula 102 proximal of the handle 100, and slots 108 cut in the outer pusher 98, allow the advancement of the outer pusher within the delivery cannula. Slots 104, 108 allow for the controlled advancement of an upstanding inner pusher control knob 106 which advances an inner pusher 128 (FIG. 10) relative to the outer delivery cannula 102. An additional slot 110 in outer pusher 98, which extends proximally from slot 108, allows advancement of the outer pusher 98 around a retaining pin that extends through the outer pusher 98, for a manipulating cannula stop collar 124 (FIG. 10), against which a manipulating cannula guide 116 (FIGS. 9, 10) stops at the appropriate depth. A suture tensioning knob 114 at the proximal end of the guide 116 allows advancement and/or rotation of the manipulating pusher 122. The manipulating pusher 122 is contained within the inner lumen of the manipulating cannula guide 116, which in turn is positioned within the inner lumen of the outer pusher 98. While the cannulae shown here are circular in cross section, other viable options include oval, rectangular, square, tapered, or irregular in shape. The illustrations generalize the stopping mechanisms of one tube sliding in relation to another, and can take any of numerous shapes. The stopping mechanism could be of many other varieties including at least a ratchet/pawl mechanism, a tongue and groove mechanism, or a threaded mechanism in addition to achieving the advancement resistance with appropriate cannulae tolerances.

FIG. 10 illustrates an exemplary mechanism involved with advancement of the inner pusher 128 which is fixed to the inner pusher guide collar 126. Perpendicular to the long axis of the inner pusher guide collar 126 is a threaded post 118 of the inner pusher control knob 106 which advances within the slot 104 in the delivery cannula 102. Rotation of the inner pusher control knob 106 locks the inner pusher guide collar 126 relative to the delivery cannula 102. The delivery cannula 102 has a downwardly extending retaining post 120 for securing the manipulating cannula stop collar 124 within the inner lumen of the delivery cannula 102 and the outer pusher 98. The slot 110 of the outer pusher 98 allows for advancement of the outer pusher past the retaining post 120 while slot 108 allows the outer pusher 98 to advance the desired length past the threaded post 118 of the inner pusher control knob 106. The manipulating pusher 122 passes through the inner lumen of the inner pusher 128 independently of the position of the inner pusher. The advancement of the manipulating pusher is controlled by the manipulating pusher guide 116 stopping against the manipulating cannula stop collar 124.

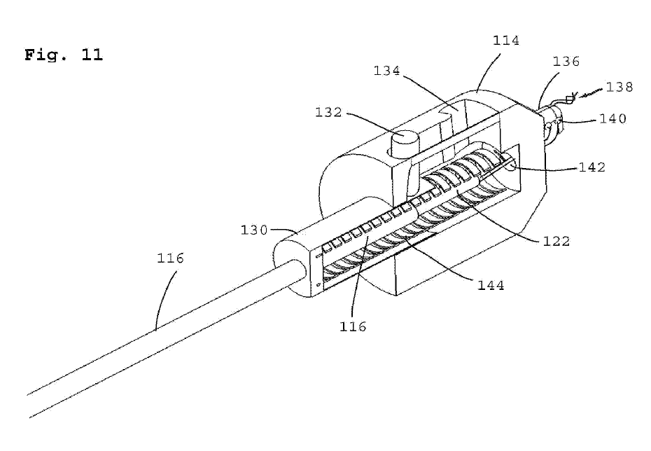

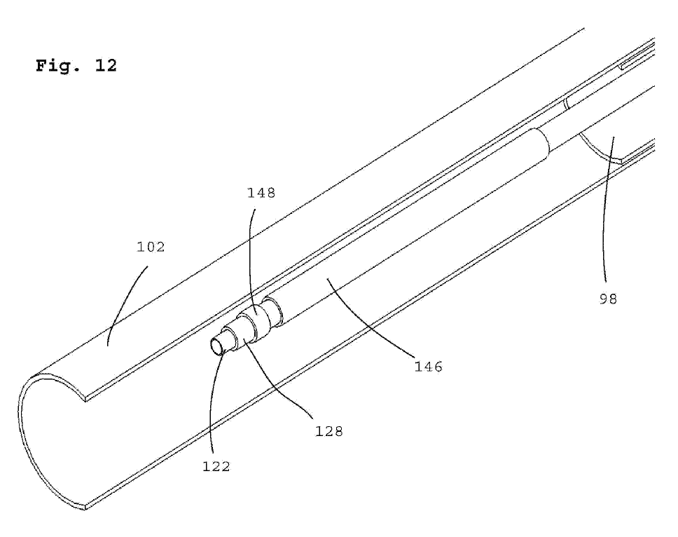

FIG. 11 illustrates the mechanism of the suture tensioning knob 114 which is made up of the female portion of the knob 114 with locking slot 134 and a laterally extending male portion 132 of the knob 130 which is fixed to the manipulating cannula guide 116. Within the female and male portion of suture tensioning knob 114 is a tensioning spring 144. The female portion of the knob 114 slides longitudinally and can rotate around the knob 130, limited by the male portion 132 riding within the slot 134. Moving the female portion of knob 114 distally relative to the knob 130 (and, thus, the guide 116) causes compression of the spring 144. The manipulating pusher 122 is fixed to the manipulating cannula guide 116 and, as shown in this illustration, is in an unlocked position, causing a separation between an outlet 142 of the female portion of the suture tensioning knob 114. The suture 138 is secured to the suture docking post 136 by passing through docking post holes 140. This mechanism is designed to allow semi-automatic engagement of the internal anchor 10 onto the manipulating pusher and/or internal pusher after deployment from the delivery cannula 102. Alternative mechanism could employ a tensioning line or manual tensioning.

FIG. 12 illustrates the distal end of the exemplary delivery system in the implant-loaded position (no implant is illustrated in FIG. 12, for clarity). The outer pusher 98 is contained within the lumen of the outer (delivery) cannula 102. The manipulating pusher 122 extends within the lumen of the inner pusher 128 which is within the lumen of a disc slider 146. The inner pusher 128 and manipulating pusher 122 in the implant-loaded position are retracted within the outer cannula 102 to allow space for the internal anchor 10 within the lumen of the outer cannula 102. The disc 50 is collapsed, in the manner of an umbrella or threefold card, and loaded within the lumen of the outer cannula 102 with the disc slider 146 extending through the disc through hole 71.

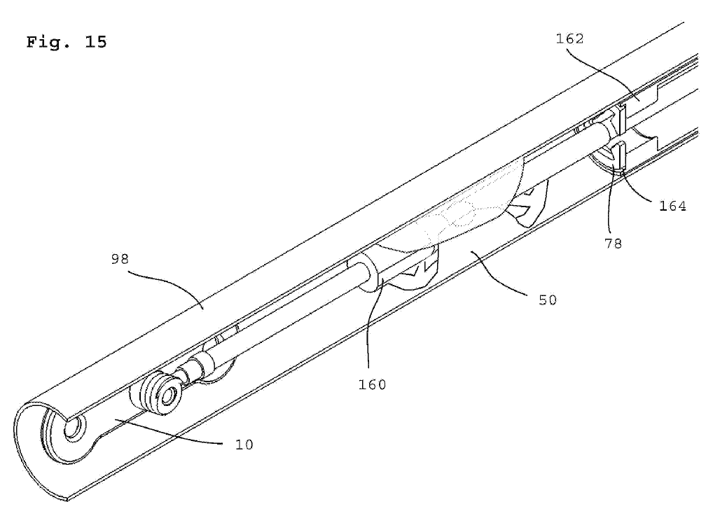

According to one exemplary embodiment, the anchor 10 is collapsed partially, in the manner of an umbrella or threefold card, and loaded within the lumen of the outer cannula 102, distal of the disc 50, with the planer component 26 extending distally away from the proximally extending tubular docking hub 21. The anchor 10 is collapsed partially as compared to the more fully collapsed disc 50 because the anchor is formed of a stiffer material than the disc, or is otherwise made more stiff than the disc. For this purpose, the structures of the anchor 10 and the disc 50 are formed of one or more materials that permit it to be folded, collapsed, or otherwise assume a smaller profile so that it can be retained within the inner lumen of the outer cannula 102. Likewise, the materials and/or structures of the anchor 10 and the disc 50 are selected so that the anchor and disc will resume the larger configuration (see FIGS. 4-6 and 8) after device deployment. Alternatively, according to another exemplary embodiment, the anchor 10 and/or the disc 50 can be fashioned in such as size and shape to allow positioning into the outer cannula 102 without being folded, collapsed, or otherwise assuming a smaller profile, such as is illustrated in FIG. 15. Differences in the modulus of elasticity and thus the ability to collapse the anchor and disc favorably affect the ability of the two members to close an anatomical defect.

The disc slider 146 allows the manipulating pusher 122 and inner pusher 128 to advance the anchor 10 out of the outer cannula 102 without advancing the disc 50. The anchor 10 is deployed out of the outer cannula 102 by the action of direct pushing from the manipulating pusher 122 and retention by the tensioning suture 138. When anchor 10 is loaded into the outer cannula 102 with the planar components 26 extending one distally and one proximally (FIG. 15), rather than a less preferred embodiment in which the anchor is loaded in a collapsed orientation with both the planar components and the hub 21 extending proximally or distally, one and then the other of the ends 12 of the planar components can be directed into the hole of an anatomical defect. In the embodiment in which the anchor is collapsed, distal advancement of the anchor 10 out of the distal end of the outer cannula results in the planar components more slowly ‘opening’ from a collapsed to an ‘open’ or planar configuration. When the docking hub 21 is fully deployed from the outer cannula 102 the female portion of the suture tensioning knob 114 is turned to place tension on the tensioning suture 138. This causes the docking hub manipulating pusher and inner pusher sleeves (16, 32) to be engaged with the manipulating pusher 122 and the inner pusher 128. Furthermore, this more controlled opening of the planar components 26 of the anchor 10 is particularly advantageously, yet still optionally, controlled by controlling the rate at which the anchor is pushed out of the outer cannula 102. Thus, the anchor 10 can be selectively positioned relative to the anatomical defect that is to be closed, e.g., positioned at least partially distal of the defect opening, opened on the distal side of that defect, then under direction of the manipulating pusher 122 and tensioning from the tensioning suture 138 be positioned on the ‘inner’ aspect of the anatomical defect in a low profile controlled fashion avoiding undue deformation of adjacent neural or neurovascular anatomy.

Advancement of the outer pusher 98 advances the disc 50 distally out of the outer cannula 102 and advances the disc slider 146 distally, due to frictional forces between on the disc slider and the disc 50, until the disc slider engages against the disc slider stop 148 mounted to the outer surface of the inner pusher 128. The disc slider stop 148 and disc slider 146 also allow for the through hole 71 to be centered on the docking post of the internal anchor 10. The retaining ring 78 also passes over the outer lumen of the disc slider 146, proximally to the disc 50, for purposes described below. In the loaded position anchor 10 is positioned at the distal end of the manipulating pusher 122 within the outer cannula 102, distal to the disc 50. The anchor 10 is temporarily held in place at the distal end of the inner pusher by the retention suture 138 extending from the suture post 136, through the inner most cannula, and attached to the anchor at the suture holes 48. The distal end of the manipulating pusher or inner pusher pushes the anchor 10 out of the delivery cannula 102 at which time the suture tensioning knob 114 can be used to ‘tension’ or pull the manipulating pusher docking sleeve 32 onto the manipulating pusher 122 to the stop 40. The inner pusher 128 extends into the internal pusher docking sleeve 16 to stop 38. Thus assembled the anchor 10 can be controlled by the delivery system for positioning within the desired anatomy.

FIG. 13 illustrates the dynamic action of the manipulating pusher 122 as it is advanced out of the inner pusher 128 during deployment and positioning of the internal anchor 10. The manipulating pusher 122 is manufactured from any suitable material, e.g., nitinol alloy, advantageously with a preformed 0 to 90 degree bend. Upon advancement out of the inner pusher 122, the nitinol alloy, a metal with memory properties, bends to its original manufactured shape. This allows for precise angulation of the anchor 10 during anatomical placement by both controlling the angle of the bend by advancement or the direction of the bend by rotation of the manipulating pusher 122 at the suture tensioning knob 114. Alternatively, the manipulating pusher 122 could be manufactured from memory shape plastics such as Pebax.

FIGS. 14a and 14b illustrate the delivery system positioned within an exemplary anatomy, in this case representing a hole in the bone and dura of the skull base. The delivery system has allowed for controlled positioning of the internal anchor 10, by manipulation of the pusher 122, on the inner side of the dura 152 and bone 150 through the cranial and dural defect represented by the hole 154. The outer pusher 98 is being advanced through the delivery cannula 102 to cause advancement of the disc 50 and retaining ring 78 over the disc slider 146 within which is the inner pusher 128 and manipulating pusher 122 (not illustrated in FIGS. 14a and 14b). With further advancement of the outer pusher 98, the disc 50 will be locked to the docking hub 21 of the internal anchor 10 by the ratchet mechanism on the internal (distal) side and the retaining ring 78 on the external (proximal) side, while the anchor 10 is temporarily held in place by the suture 138 extending from the suture post 136, through the innermost cannula, and attached to the anchor at the suture holes 48. The tolerance of the fit between the manipulating pusher docking sleeve 32 into the manipulating pusher 122 and the inner pusher docking sleeve 16 into inner pusher 128 also affords control of the anchor 10. This will aid in closing the anatomical defect in a low profile fashion. Once the ring 78 has captured onto the ratchet teeth 22 the components of the device are positioned to close the anatomical defect. The outer pusher 98 can compress the disc 50 onto the docking hub 21 to gain favorable advantage in creating a seal against adjacent anatomical structures.

The outer disc, docking hub, or retaining ring may have the additional mechanism of holding in place additional tissue(s), graft, or glues. This could be accomplished by juxtaposing anatomical or other tissue between the device components. Further modifications such as hooks, tabs, suture, or a plurality of discs, partial or complete could modify the device for potential useful function in closing anatomical defects. These additional structures could be positioned on the inner, outer, or perimeter of the disc for favorable use in capturing adjacent anatomical tissues.

FIG. 15 illustrates a longitudinal sectional view of distal portions of an exemplary embodiment of a system, partially described above, in which the anchor 10 and the disc 50 are mounted within the outer cannula 102. The anchor 10 is not collapsed, but the disc is longitudinally collapsed, viewed from the outer surface 68. In this exemplary embodiment, the anchor 10 is positioned within the lumen of the outer cannula in a longitudinal orientation, that is, with the planar components 26 extending longitudinally, with the suture or wire 138 only loosely holding the anchor to the distal end of the inner pusher and manipulator pusher. When the anchor 10 is pushed out of the outer cannula 102, the suture or wire 138 can be pulled proximally (tensioned), which seats the manipulator pusher and the inner pusher in the hub 21, as described herein.

Similarly, the disc 50 is folded like a three-fold-card or a “taco shell”, that is, somewhat wrapped around the disc slider. An alternative disc slider 160 is illustrated, which is similar to disc slider 146 with the addition of an enlarged, cylindrical distal portion and a frustoconical proximal portion, obscured in the drawing by the disc 50. Further optionally, the retaining ring 78 is mounted in a shoulder 164 formed in a thickened distal end portion 162 of the outer pusher 98, which assists in holding the ring in place until deployment, as otherwise described herein.

The implants of the present invention are most favorably made of biodegradable or bioabsorbable material. This material can be polymeric, oligomeric, or monomeric materials. The monomers are often joined at an amide linkage creating poly amino acids. When the implant is formed of material that biodegrades it is favorable to select a material composition that will allow for the desired anatomy to close over with native tissue by the natural healing process prior to significant degradation of the implant. The degradation rate can be modified by changing the degree of polymerization and/or modifying the amount of crosslinking between chains. The foregoing is not intended to limit the materials within the scope of this invention, but to highlight favorable characteristics.

Examples of preferred materials include biodegradable polymers polycaprolactones, poly(amino acids), polyanhydrides, aliphatic polyesters, polyothroesteres, polylactic acid including either D, L and D/L isomers, poly(lactide-co-glycolide), and copolymers of polylactide and caprolactones, and poly-4-hydroxybuterate. A preferred example for the outer disc is a copolymer of 70:30 poly(D/L) lactide: caprolactone. Further the implants could be composed of or coated with materials such as polyethylene glycol which swells on contact with fluids. This creates an additional mechanism by which the anchor 10 and/or the disc 50 can close the anatomical defect.

A favorable benefit of devices embodying principles of the present invention is the ease of manufacturing synthetic implants. The implants can be formed by a process of injection molding, blow molding, or extrusion. A favorable modification to the materials of the device would be the addition of a hydrogel, expandable sponge, or materials with hydrophilic or hydrophobic properties to the surface of the implant. The anchor 10 could be made entirely from an expandable material.

By way of another non-limiting example, a delivery apparatus includes a 2 to 20 mm diameter rigid outer delivery port with a straight or curved tip. Through this port a semi-rigid guide wire with an outer semi-rigid sheath is inserted. The semi-rigid guide wire is made up of an inner semi-rigid wire and an outer semi-rigid sheath. The docking pin of the device inner component is docked at the semi-rigid guide wire. The guide wire outer sheath is then advanced over the inner semi-rigid guide wire to the base of the docking pin. This secures the device to the semi-rigid guide wire, allowing the device to be controlled by the guide wire. The device is opened to allow loading into the delivery port. Small loops at the ends of the struts on the inner and outer device components allow for securing of the tension modulating sutures.

The flexibility offered in the diameter of the delivery port in this application (minimally invasive dural, bone, or ligamentous closure) allows for the variability in the dimensions of various implant devices to be deployed. This feature allows critical differences between this device and alternative devices that are delivered through much smaller transluminal endovascular catheters.

The framework of the implant is in the closed position when loaded into the delivery cannula. It is deployed from the delivery cannula in a controlled, non-automatic fashion. This is performed by bracing the struts on the side walls of the delivery cannula such that the inner component of the device is partially opened prior to being fully deployed and by use of tension modulating sutures attached to the struts which extend proximally for control by the practitioner. The outer component is deployed in a controlled fashion by use of the semi-rigid guide wire and the tension modulating sutures. Both the inner and outer components of the device are deployed under direct optical visualization.

Exemplary steps for using such an alternative embodiment include: advance the device to deploy a first component of the implant; advance another portion of the device to actuate manipulation of the implant, with nitinol or other flexible tube or rod material; turn a suture retraction knob to dock a component of the implant onto a manipulation tube; advance the manipulation tube to a desired distance, to position the implant; advance the device to straighten the manipulation tube, thus preparing the first component of the implant to receive a second component of the implant; advance an outer pusher to deploy an outer (second) component of the implant; advance the device to lock components of the implant together; release a suture retraction knob from the suture to release the implant device from the delivery device. A series of tubes one inside another slide in such a way as to allow control, manipulation, or other precise movements of an implanted device.

While the invention has been described in detail with reference to exemplary embodiments thereof, it will be apparent to one skilled in the art that various changes can be made, and equivalents employed, without departing from the scope of the invention. The foregoing description of the preferred embodiments of the invention has been presented for purposes of illustration and description. It is not intended to be exhaustive or to limit the invention to the precise form disclosed, and modifications and variations are possible in light of the above teachings or may be acquired from practice of the invention. The embodiments were chosen and described in order to explain the principles of the invention and its practical application to enable one skilled in the art to utilize the invention in various embodiments as are suited to the particular use contemplated. It is intended that the scope of the invention be defined by the claims appended hereto, and their equivalents. The entirety of each of the aforementioned documents is incorporated by reference herein.

What is claimed is:

1. A system for closure of anatomical openings in a patient, comprising:
an anchor including a first locking element extending along a longitudinal direction and at least two lateral elements extending at least partially laterally from the longitudinal direction;
a flexible sealing disc including a sealing membrane and a hole through said disc sized to permit passage of the first locking element therethrough; and
a retaining ring including a second locking element configured and arranged to receive the first locking element and lockingly retain the ring to the anchor.

2. A system in accordance with claim 1, wherein the at least two lateral elements extend orthogonally from the first locking element.

3. A system in accordance with claim 1, wherein the anchor, the disc, and the ring are formed of one or more bioresorbable materials.

4. A system in accordance with claim 1, wherein the disc is positioned between the ring and the anchor, with the first locking element extending through the disc hole and into locking engagement with the second locking element.

5. A system in accordance with claim 1, wherein the first locking element comprises a tube having an exterior surface including at least one ratchet tooth

6. A system in accordance with claim 1, wherein the first locking element further comprises a central bore having proximal and distal openings.

7. A system in accordance with claim 6, wherein the first locking element further comprises a constriction in the central bore configured and arranged to retain a tension wire thereon.

8. A system in accordance with claim 1, wherein at least one of the at least two lateral elements comprises a planar portion attached to the first locking element and an enlarged end.

9. A system in accordance with claim 1, wherein said at least one lateral element further comprises a hole extending therethrough.

10. A system in accordance with claim 1, wherein at least one lateral element is formed of a material selected to permit the at least one lateral element to be bent toward the longitudinal axis.

11. A system in accordance with claim 1, wherein the disc comprises:
a central hub through which said hole extends; and
an outer rim;
wherein said membrane extends between said central hub and said outer rim.

12. A system in accordance with claim 11, wherein the disc further comprises a plurality of struts extending between the central hub and the outer rim.

13. A system in accordance with claim 12, wherein the membrane is positioned on one longitudinal side of the struts.

14. A system in accordance with claim 12, wherein the membrane is positioned on the proximal side of the struts.

15. A system in accordance with claim 12, wherein at least the membrane and the plurality of struts are formed of materials selected to permit the membrane and the plurality of struts to be collapsed.

16. A system in accordance with claim 1, wherein the disc is dome shaped including an outer convex surface, an inner concave surface, and a lip joining the outer and inner surfaces.

17. A system in accordance with claim 16, wherein the outer rim forms said lip, and the central hub is positioned opposite the outer rim.

18. A system in accordance with claim 16, wherein the struts are curved along the dome shape.

19. A system in accordance with claim 1, wherein the retaining ring comprises:
a ring with a hole; and
at least one pawl radially inwardly extending on the ring, the at least one pawl configured and arranged to engage and form a lock with said first locking element when said first locking element is at least partially positioned in said ring hole.

20. A system in accordance with claim 19, wherein the at least one pawl comprises a plurality of pawls circumferentially spaced around said ring.

21. A system in accordance with claim 1, wherein the retaining ring further comprises a radially outer surface and at least one groove in the radially outer surface.

22. A system in accordance with claim 1, further comprising:
an outer cannula having a lumen extending therethrough;
an outer pusher having a distal end with a lip and a lumen extending therethrough, the outer pusher positioned in the lumen of the outer cannula;
an inner pusher having a distal end and a lumen extending therethrough, the inner pusher positioned in the lumen of the outer pusher;
a manipulating pusher having a distal end and being positioned in the lumen of the inner pusher;
wherein the ring is positioned in the outer pusher distal end lip;
wherein the disc is positioned in the outer cannula lumen distal of the ring; and
wherein the anchor is positioned at the manipulating pusher distal end and distal of the disc.

23. A method for closing an anatomical defect in a patient, the defect including a hole with a lateral dimension, the method comprising:
inserting an anchor through said hole, a portion of the anchor extending back through the hole;
attaching a seal to said anchor portion, said seal extending laterally farther than said defect hole lateral dimension.
